(19) United States
(12) Patent Application Publication
(10) Pub. No.: US 2008/0262053 A1
(43) Pub. Date: Oct. 23, 2008

(54) Use of Pramipexole for Treating Moderate to Severe Restless Legs Syndrome (Rls)

(76) Inventor: Juergen Reess, Ulm (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/090,428

(22) PCT Filed: Oct. 16, 2006
(86) PCT No.: PCT/EP2006/067408

§ 371 Date: May 19, 2008

Related U.S. Application Data
(60) Provisional application No. 60/727,985, filed on Oct. 18, 2005.

(51) Int. Cl. A61K 31/428; A61P 25/00

(52) U.S. Cl. 514/367

(57) ABSTRACT

The invention relates to the use of 2-amino-6-n-propylamino-4,5,6,7-tetrahydrobenzo-thiazole, the (+)- or (−)-enantiomer thereof or the pharmacologically acceptable salts thereof for treating moderate to severe Restless Legs Syndrome (RLS).

The invention relates to the use of 2-amino-6-n-propylamino-4,5,6,7-tetrahydrobenzo-thiazole, the (+)- or (−)-enantiomer thereof or the pharmacologically acceptable salts thereof for the treatment of moderate to severe Restless Legs Syndrome (RLS).

Pramipexole-2-amino-6-n-propylamino-4,5,6,7-tetrahydrobenzo-thiazole dihydrochloride—is a dopamine—D2/D3 agonist the synthesis of which is described in European Patent 186 087. Pramipexole is known primarily for the treatment of idiopathic Parkinson's, as a monotherapy or in conjunction with levodopa. It is known from German Patent Application DE 38 43 227 that pramipexole lowers the prolactin serum level, and it is also known from German Patent Application DE 39 33 738 to use pramipexole for lowering high TSH levels. Transdermal application is described in U.S. Pat. No. 51,112,842, and WO Patent Application PCT/EP93/03389 describes the use of pramipexole as an antidepressant. WO9831362 proposes using pramipexole for treating Restless Leg Syndrome.

Restless Legs Syndrome (synonyms: RLS, anxietas tibiarum, Wittmaack-Ekbom syndrome) is a neurological ailment characterised by an uncontrolled compulsion to move the legs. Usually, accompanying symptoms include unpleasant and sometimes painful sensations in the legs such as prickling, dragging pains, tearing, itching, burning, cramps etc. RLS is estimated to affect up to ten percent of people between 30 and 79 years old. The symptoms get worse in the evening and at night, with the result that those affected often additionally suffer from sleep disorders. The consequences are tiredness and irritability in the daytime with the corresponding consequences for daily work and social life.

Although RLS is widely prevalent in the population, the diseases is still largely unknown or undiagnosed. The diseases affects the quality of life of millions of people worldwide. Those affected generally perceive its influence on their daily lives as being more serious than the influence of chronic complaints such as high blood pressure, diabetes or heart disease.

If the patient's sleep or quality of life is increasingly restricted by RLS or the patients are suffering from daytime fatigue, treatment is indicated. A need for treatment generally starts at the age of 40 to 50. In therapeutic studies, monotherapies with dopamine agonists, opiates, benzodiazepines, carbamazepine, clonidine or levodopa (L-DOPA) combined with a dopadecarboxylase inhibitor demonstrated varying degrees of success. The most work has been done on the use of L-DOPA in RLS. When used in long-term therapy there is a significant reduction in pain, with an improvement in sleep or quality of life. However, the disadvantage of L-DOPA therapy is that in many patients the effect wears off and/or the RLS pains are displaced to the morning (rebound) or afternoon (augmentation).

Large-scale randomised clinical trials have now shown that pramipexole leads to a rapid improvement in the RLS symptoms in patients suffering from moderate to severe RLS. The expression moderate to severe RLS is used when patients reach a points score of >15 on the international RLS scale. The scale runs from 0 (no RLS symptoms) to 40 (severest form of RLS). Thus, in moderate to severe RLS, the symptoms usually occur more than twice a week.

After only one week, treated patients report significant improvements in all areas of symptoms, such as quality of sleep, restlessness of the legs, etc.

For the purposes of the present invention the term improvements in the symptoms of RLS means that pramipexole can reduce the points score on the RLSRS scale after 3 weeks of treatment by at least 10 points, preferably at least 12 points, more preferably at least 14 and most preferably at least 15 points and this score is also maintained. Thus, it was found that pramipexole is extremely efficient and well tolerated in single doses in the form of tablets containing 0.1 to 1.5 mg, preferably 0.1 to 1 mg, more preferably 0.125 mg to 0.75 mg of active substance, taken once a day. When pramipexole is administered continuously, the improvement in the symptoms in RLS patients lasts for at least 6 months or more (lasting effect).

Preferred daily doses are between 0.08 and 1 mg. The following daily doses and all the intermediate values are preferred: 0.088 mg, 0.18 mg, 0.125 mg, 0.25 mg; 0.35 mg, 0.5 mg; 0.7 mg, 0.75 mg.

Most particularly preferred are daily doses of from 0.18 mg to 0.5 mg, more preferably 0.25 mg to 0.5 mg.

This improvement is also reflected in the subjective impressions of the treated patients, the majority of whom are aware of a great or very great improvement.

Open trials showed that the lasting clinical effects persist even after 12 months.

Regarding the international RLS scale, reference is made to:
1. Allen, R. P., Picchietti, D., Hening, W. A., Trenkwalder, Allen, R. P., Picchietti, D., Hening, W. A., Trenkwalder, C., Walters, A. S., Montplaisir, J.: Restless legs syndrome: diagnostic criteria, special considerations, and epidemiology. A report from the restless legs syndrome diagnosis and epidemiology workshop at the National Institutes of Health. Sleep Med. 4 (2003), 101-120.
2. Walters, A. S. and the International Restless Legs Syndrome Study Group: Towards a better definition of the Restless Legs Syndrome. Mov. Disord. 10 (1995), 634-642.

Pramipexole is preferably used as a free base, 2-amino-6-n-propylamino-4,5,6,7-tetrahydrobenzothiazoline, or in the form of a pharmacologically acceptable salt. Particularly preferred are salts with hydrochloric acid, especially the dihydrochloride.

Apart from the tablets already mentioned, other galenic preparations are known from the prior art, such as for example plain or coated tablets, suppositories, solutions for injection or drops.

### CLINICAL INVESTIGATION

The lasting effect of pramipexole on patients with RLS was investigated in a multicentre, placebo-controlled, double-blind randomised trial in Germany. All the patients included in the trial had first taken pramipexole for 6 months before being started on a randomised double-blind controlled withdrawal phase.

All the patients who showed the treatment effects defined above after the 6-months treatment were included in the controlled second phase of the trial.

150 patients with idiopathic RLS who had responded to the treatment were selected on a random basis and were treated for a further 3 months with placebo (n=72) or pramipexole (n=78) in individualised doses of 0.125 to 0.75 mg. It was expected that in the placebo group the RLS symptoms would return or intensify, while in the pramipexole group the patients would continue to benefit from the treatment already started. The severity of the symptoms was determined by means of the difference between the time of randomisation after 6 months and the results after the end of the trial, according to the international RLS scale. The results of 147 patients were evaluated. The patients were on average 59.6 years old, 72.8% were women, the symptoms had been present on average for 5.6 years. The severity of the RLS symptoms increased significantly more in the placebo group than in the pramipexole group over the trial period. The standard deviation from the base line according to the international RLS scale was +14.86 for the placebo group and +2.03 for the pramipexole group. The trial shows that continuous treatment with pramipexole even over 6 months or more leads to a lasting improvement in the RLS symptoms.

1. A method for treating moderate to severe Restless Legs Syndrome comprising administering to a patient a therapeutically effective amount of 2-amino-6-n-propylamino-4,5,6,7-tetrahydrobenzo-thiazole, the (+)- or (−)-enantiomer thereof, or a pharmacologically acceptable salt thereof, for treating moderate to severe Restless Legs Syndrome (RLS) in said patient.

2. A method according to claim 1, wherein moderate to severe RLS is present in the patient when the patient is an affected, untreated patient having a point score of >15 on the international RLS scale.

3. A method according to claim 1, wherein moderate to severe RLS is present in the patient when RLS symptoms occur more than twice a week in the patient.

4. A method for improving the symptoms of RLS in a patient for a period of 6 months or more comprising administering to a patient 2-amino-6-n-propylamino-4,5,6,7-tetrahydrobenzo-thiazole, the (+)- or (−)-enantiomer thereof, or a pharmacologically acceptable salts thereof, in a dosage of from 0.1 to 1.5 mg once a day over the same period.

5. A method according to claim 4, wherein the RLS is moderate to severe RLS, and the patient is an affected, untreated patient having a point score of >15 on the international RLS scale.

6. A method according to claim 4, wherein the RLS is moderate to severe RLS and RLS symptoms occur more than twice a week in the patient.

7. A method for improving the symptoms of RLS in a patient within a period of 1 week comprising administering to a patient 2-amino-6-n-propylamino-4,5,6,7-tetrahydrobenzo-thiazole, the (+)- or (−)-enantiomer thereof, or a pharmacologically acceptable salts thereof, in a dosage of from 0.1 to 1 mg once a day over the same period.

8. A method according to claim 7, wherein the RLS is moderate to severe RLS, and the patient is an affected, untreated patient having a point score of >15 on the international RLS scale.

9. A method according to claim 7, wherein the RLS is moderate to severe RLS, and RLS symptoms occur more than twice a week in the patient.

10. A method according to claim 2, wherein the patient has a point score of >20 on the international RLS scale.

11. A method according to claim 4, wherein the period is at least 7 months.

12. A method according to claim 4, wherein the period is at least 8 months.

13. A method according to claim 5, wherein the patient has a point score of >20 on the international RLS scale.

14. A method according to claim 8, wherein the patient has a point score of >20 on the international RLS scale.
